(19) United States
(12) Patent Application Publication
(10) Pub. No.: US 2013/0060209 A1
(43) Pub. Date: Mar. 7, 2013

(54) Athletic Tape

(71) Applicants: Parker Campbell Tyler, Salt Lake City, UT (US); Jared Turley, Salt Lake City, UT (US)

(72) Inventors: Parker Campbell Tyler, Salt Lake City, UT (US); Jared Turley, Salt Lake City, UT (US)

(21) Appl. No.: 13/469,836

(22) Filed: May 11, 2012

Related U.S. Application Data
(60) Provisional application No. 61/518,828, filed on May 12, 2011.

(51) Int. Cl. A61M 35/00

(52) U.S. Cl. 604/307

(57) ABSTRACT

Adhesive tape for use in connection with treatment of injuries is described. Tape may be configured to relieve pressure and increase circulation at the injury site, as well as deliver a topical drug solution, including NSAID solutions. Elements such as drug solutions, adhesive, water soluble gels, and so forth may be disposed in patterns configured to increase efficacy and user comfort.

### RELATED APPLICATIONS

This application claims priority to U.S. Provisional Application 61/518,828 filed on May 12, 2011 titled Transdermal NSAID Kinesiology Tape, which is incorporated herein by reference in its entirety.

### TECHNICAL FIELD

The present disclosure relates generally to tapes, wraps, and bandages configured to treat soft tissue injuries, including sports injuries. More specifically, the present disclosure relates to athletic tape that may be configured as a kinesiology-type tape and/or may comprise one or more layers of therapeutic compounds, such as non-steroidal anti-inflammatory drugs (NSAIDs), which may be configured for transdermal delivery.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a top schematic view of one embodiment of a fabric for use as a base layer of athletic tape.

FIG. 1A is a close-up view of the base layer of FIG. 1 showing a detail view of the weave pattern of this embodiment.

FIG. 2 is a top schematic view of a second embodiment of a fabric for use as a base layer of athletic tape.

FIG. 2A is a close-up view of the base layer of FIG. 2 showing a detailed view of the weave pattern of this embodiment.

FIG. 3 is a side schematic view showing a base layer and an adhesive layer, which may be provided in connection with other layers not shown in this view.

FIG. 4 is a side schematic view showing a base layer and a drug solution layer, which may be provided in connection with other layers not shown in this view.

FIG. 5 is a side schematic view showing a base layer and water channels, which may be provided in connection with other layers not shown in this view.

FIG. 6 is a side schematic view showing a base layer and water channels which include a water soluble gel, which may be provided in connection with other layers not shown in this view.

FIG. 7 is a side schematic view of a base layer with a pattern of additional layers of adhesive, drug solution, adhesive, and a water channel, respectively, disposed thereon.

FIG. 7A is a bottom schematic view of a portion of the base layer of FIG. 7 showing the relative placement of the additional layers in chevron-shaped strips across the base layer.

FIG. 8 is a side schematic view of a base layer with a pattern of additional layers of adhesive, a water channel, and drug solution, respectively, disposed thereon.

FIG. 8A is a bottom schematic view of a portion of the base layer of FIG. 8 showing the relative placement of the additional layers in parallel strips across the base layer.

FIG. 9 is a bottom schematic view of a base layer, with additional layers of drug solution, adhesive, water soluble gel, adhesive, drug solution, and a water channel, respectively, disposed thereon.

FIG. 10 is a bottom schematic view of a base layer, with additional layers of drug solution, adhesive, water soluble gel, drug solution, adhesive, and a water channel, respectively, deposited thereon.

FIG. 11 is a bottom schematic view of a base layer with additional layers of adhesive and drug solution deposited in a dotted pattern on a field of water channels, portions of which contain a water soluble gel.

### DETAILED DESCRIPTION

Athletic tape may be configured to treat soft tissue injuries, including sports injuries. For example, athletic tape may be used to treat injuries such as sprains, strains, knee injuries, swollen muscles, contusions, tendonitis, and stress injuries. Some soft tissue injuries may result in swelling, pain, limited range of motion, and other ill effects. Athletic tape may be used to provide support to such injuries, aid in healing, and reduce the risk of re-injury. Further, athletic tape may be used to provide support to minimize the likelihood of an initial injury. Similarly, such tape may reduce the risk of re-injury or aggravation when an athlete continues to participate in athletic activities while a soft tissue injury is still healing or just after an injury has healed.

The current disclosure is relevant to the treatment of many soft tissue and sport related injuries, including treatment of the first phase, or acute inflammatory phase, of such injuries. The acute inflammatory phase of an injury may be characterized by pain, swelling, and immobilization.

In some embodiments, the athletic tape disclosed herein may be a kinesiology tape. Kinesiology tape may be designed to pull or lift skin off of the muscles surrounding an injury, thereby facilitating the circulation of both blood and lymphatic fluid. As the skin is lifted off the muscle, pain receptors may be relieved from pressure that was applied due to inflammation.

Kinesiology tapes may be placed on the body in particular or specific locations or patterns to address specific injuries or to provide a specific benefit. The art of kinesiology taping is designed to provide a wide range of support for a variety of injuries. When used as a single piece, kinesiology tape may provide elasticity similar to that of skin. As a result, small amounts of tape may not significantly limit a user's range of motion. The tape may be placed in particular patterns to align injured muscles and tendons into proper position, to prevent further injury and to relieve force around inflamed tissue. In instances where a large amount of support is desirable, multiple pieces of tape can be used. In some circumstances, this use of the tape may tend to limit the range of motion of the injured area. Hence, certain taping patterns can constrain injured areas to maintain proper functional movements. Additionally, different amounts of stretch can be applied to the tape prior to placement, to create different amounts of force on or around the muscle and injury. The tape may be designed to be comfortable for long term wear up to 24 hours and may be usable in aqueous environments.

The kinesiology tape of this disclosure may enable a topically applied drug treatment. Specifically, in some embodiments, a drug configured for transdermal delivery may be deposited on portions of one or more surfaces of the athletic tape such that it is delivered after application of the tape to a user. The drug may be a prescription or non-prescription drug selected from pain relievers, anti-inflammatory compounds, and/or other drugs useful in the treatment of athletic injuries singly or in combination. In some specific examples of the invention, the drug incorporated may be a non-steroidal anti-inflammatory drug (NSAID) adapted to be topically administered to decrease swelling and pain related to, for example, a soft tissue injury. As further detailed below, in some embodiments, a drug such as an NSAID may be deposited on the bottom surface of the adhesive kinesiology tape disclosed herein wherein the tape may be configured to stabilize, support, and relieve pressure on an injury site.

It will be readily understood that the components of the embodiments, as generally described and illustrated in the figures herein, could be arranged and designed in a variety of configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

The phrases “connected to,” “coupled to,” and “in communication with” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

A fabric layer may form a base or substrate layer of athletic tape. A fabric layer may define a top surface, or the surface configured to face outwardly from an individual when the tape is applied to the individual, and a bottom surface, or the surface configured to face or contact an individual's skin when the tape is applied to the individual. Adhesive configured to couple the tape to the individual's skin may be deposited on the bottom surface of the base layer.

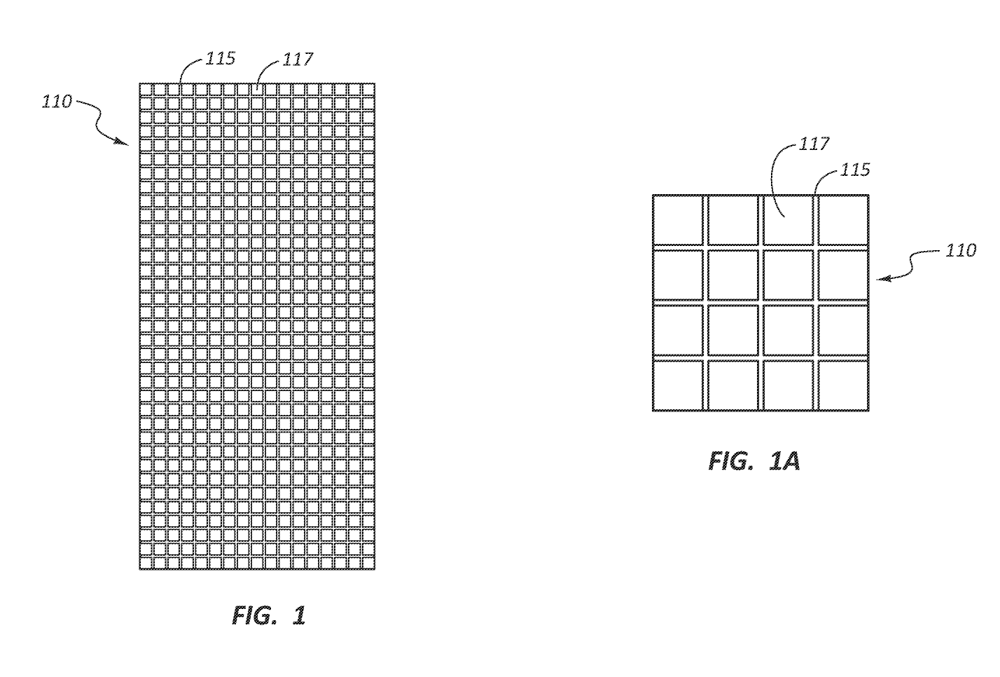

FIG. 1 is a top schematic view of one embodiment of a fabric weave for use as a base layer 110 of athletic tape. FIG. 1 is “schematic” in nature in that it illustrates how individual fibers 115 may be positioned relative to each other to form a weave or mat, while not being intended to show the actual size of fibers 115 or the pores 117 between such fibers. FIG. 1 likewise does not necessarily show actual proportions between fiber 115 size or diameter and the size of the pores 117 between such fibers 115. FIG. 1A is a close-up view of the base layer 110 of FIG. 1, further illustrating the arrangement of fibers 115 and the positioning of the pores 117. As illustrated in FIGS. 1 and 1A, the fibers 115 may be positioned at right angles to each other to form a base layer 110.

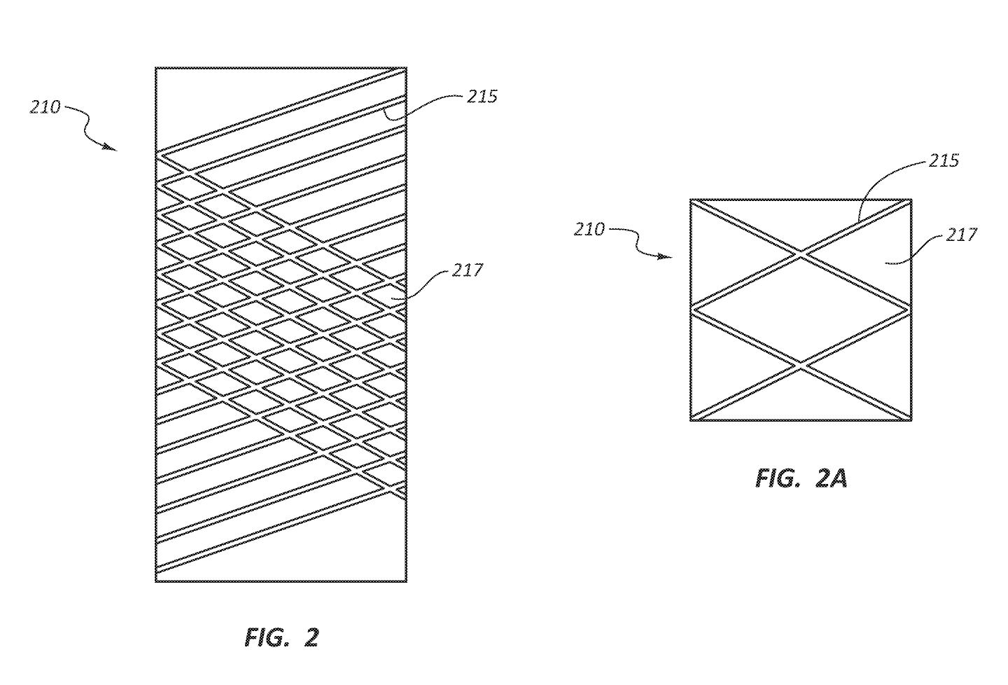

FIG. 2 is a top schematic view of a second embodiment of a fabric weave for use as a base layer 210 of athletic tape. In the embodiment of FIG. 2 the fibers 215 are positioned at an angle relative to each other. FIG. 2 is not intended to illustrate a precise angle between fibers 215; angles both larger and smaller than the illustrated angle are within the scope of the current disclosure. FIG. 2A is a close-up view showing the spatial relationship between adjacent fibers 215. Like the embodiment of FIG. 1, the fibers 215 of FIGS. 2 and 2A are arranged such that pores 217 are posited between fibers 215.

Referring to the embodiments of FIGS. 1 and 2, the base layer 110, 210 may be configured to optimize the directionality of forces across the tape. In other words, the fibers 115, 215 may be positioned based on the intended use of the tape, with some arrangements providing more strength along the length of the tape while others provide more strength across the width of the tape. In some embodiments the desired strength of the base layer 110, 210 in either direction may be balanced against the desired elasticity of the base layer 110, 210 in either direction.

In some embodiments, the fibers 115, 215 may be woven together to form the base layer 110, 210. Similarly, in some embodiments the fibers 115, 215 may be melted, fused, glued, or otherwise sealed along the edges to prevent fraying of the base layer 110, 210. The fibers 115, 215 may comprise a variety of fabrics, including, for example, naturally-based fabrics such as cotton and/or synthetic fabrics such as polyester or polyester-based fabrics. In some embodiments, a base layer 110, 210 may be composed of multiple types of fabrics used in connection with each other.

Furthermore, in some embodiments the fibers 115, 215 may be arranged such that the tape has an elasticity of between about 100% and about 180%. In other words, the fabric can stretch to 100%-180% of its length or width without tearing, and tends to return to its shape after being stretched. In some embodiments, the tape may have an elasticity of between about 120% and about 140%. The pores 117, 217 may be configured to promote breathability, that is, gas or vapor exchange across the base layer 110, 210. In some embodiments the base layer 110, 210 may comprise multiple individual layers of fibers 115, 215 layered on top of each other. The porosity and permeability of the base layer 110, 210 may be controlled by both the size of the pores 117, 217 in each individual layer and the total thickness of the layers together.

The porosity and breathability of the base layer 110, 210 may be configured to allow moisture on an individual's skin, for example sweat, to evaporate and escape through the base layer 110, 210. In some embodiments the base layer 110, 210 may be sufficiently porous to allow an individual to comfortably wear the tape for an extended period of time, for example 24 hours or more.

Layers of additional substances may be applied to the base layer 110, 120 for adhesion, drug delivery, breathability, water resistance, and so on. As used herein, an “additional layer” refers to any layer applied to a base layer for any purpose. Further, the additional layers described herein may only be deposited on portions of the base layer, for example in strips across the width of the base layer. Thus, an “additional layer” does not necessary cover the entire surface of the base layer. Furthermore, as used herein, a single “additional layer” refers to a single strip, portion, or segment. Thus, in embodiments where the additional layers are disposed in horizontal strips across the width of a base layer, each strip comprises an additional layer.

Moreover, as used herein, the term “additional layer” encompasses portions of the tape which are left open in connection with “additional layers” which include depositing a substance on the tape. Specifically, in some instances, kinesiology tape, as disclosed herein, may have strips or segments on the back of the tape which are surrounded by layers such as adhesive or drug solution, but the strips themselves are open. In some embodiments, for example, tape may have alternating adhesive and open strips to allow for breathability and comfort, as opposed to covering the entire base layer with adhesive or another substance. These open segments may be designed with the same, or analogous, shapes, sizes, and patterns as the layers of adhesive, drug solution, gel, and so forth. Thus, for convenience herein, portions of the base layer which are left open without depositing an additional substance thereon as well as strips or portions with a substance deposited thereon, are collectively referred to as “additional layers.” Disclosure relevant to any additional layer, for example, position, size, shape, and so on, may therefore be applied to any other layer or to the open portions, even those these portions do not include a substance deposited thereon.

In some embodiments, the base layer 110, 210 may be configured to resist leakage of materials comprising the additional layers across the base layer 110, 210. In other words, the base layer 110, 210 may be configured to be impermeable with respect to the material comprising one or more additional layers. Further, in some embodiments, portions of the base layer 110, 210 may be configured as impermeable to certain elements while other portions are not. For example, portions of the base layer which contact the drug solution layers (430 of FIG. 4) may be impermeable to the drug solution while other portions of the base layer 110, 210 are not so configured. For example, a base layer 110, 210 may comprise a polymer backing disposed opposite drug solution layers (430 of FIG. 4) while other portions of the base layer 110, 210 remain without a backing. Some embodiments may be so configured to limit the extent to which the backing reduces air and vapor flow across the base layer 110, 210, while containing the drug solution (430 of FIG. 4). Any backing or lack thereof may be configured for use in connection with any of the additional layers described herein.

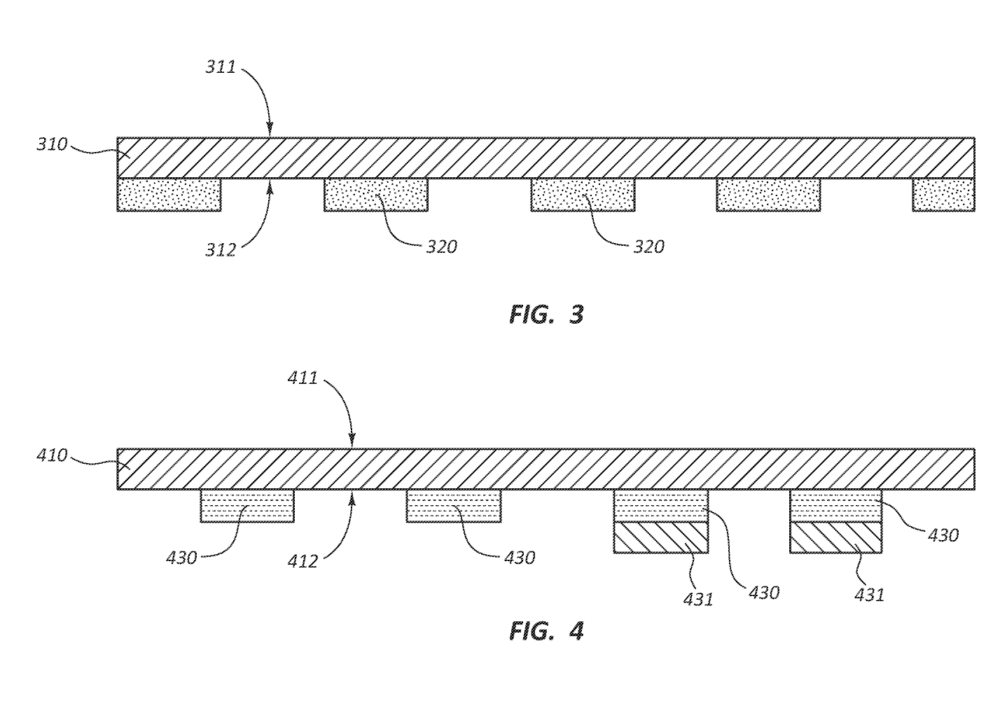

FIGS. 3-6 are schematic views showing a number of additional layers deposited different base layers. These figures illustrate how each additional layer may be used in connection with kinesiology tape as described herein. However, these figures should be understood as illustrating potential building blocks of different embodiments of kinesiology tape, as described herein, rather than necessarily viewing each as a complete embodiment. Each additional layer is discussed in connection with differing figures for convenience in describing each layer and to illustrate how any layer herein described may be used in combination with any other layer. Furthermore, in these and subsequent figures, the figures are not intended to represent an entire piece of kinesiology tape, but rather to schematically illustrate designs and patterns. Thus, actual embodiments may contain many more addition layers, including repeating patterns of layers as illustrated, than the illustrated embodiments.

FIG. 3 is a side schematic view of a base layer 310 defining a top surface 311 and a bottom surface 312. In the embodiment of FIG. 3, an adhesive layer 320 is deposited on portions of the bottom surface 312 of the base layer 310. The adhesive layer 320 may be configured to adhere the base layer 310 to the skin of a user. Any adhesive, including medical grade acrylic adhesives, may be used in connection with the base layer 310. In some embodiments, the adhesive may be hypoallergenic and/or latex free. Further, the adhesive layer 320 may be configured to allow a user to wear the tape for an extended period of time without it becoming loose. Factors such as durability, tack, peel adhesion, sheer strength, and layer pattern may be considered in selecting an appropriate adhesive. Moreover, the adhesive layer 320 may be configured to maintain its properties while in the presence of moisture. In some embodiments, heat or pressure sensitive adhesive may be used.

FIG. 4 is a side schematic view of a base layer 410 defining a top surface 411 and a bottom surface 412. In the embodiment of FIG. 4, a layer of drug solution 430 is deposited on portions of the bottom surface 412 of the base layer 410.

FIG. 4 illustrates a base layer 410 and a drug solution layer 430 while FIG. 3 illustrates a base layer 310 and an adhesive layer 320. Certain elements and subcomponents of each of these embodiments can, in certain respects, resemble elements and subcomponents of the other. It will be appreciated that all the illustrated embodiments may have analogous features. Accordingly, the relevant descriptions of such features apply equally to the features of each embodiment. Any suitable combination of the features, and variations of the same, described with respect to the components illustrated in FIG. 3, can be employed with the components of FIG. 4, and vice versa. Furthermore, analogous or substantially analogous features shown in one figure may or may not be designated by a reference numeral regardless of whether the analogous feature is so designated in the analogous figure.

This pattern of disclosure applies equally to further embodiments and components described herein. For example, any of the base layers described herein (for example those recited in connection with FIGS. 1 and 2) may be used in connection with any of the additional layers deposited on the base layers described in connection with FIGS. 3, 4, and so on. Furthermore, any of the additional layers, and patterns of depositing the same, described in connection with any figure, may be relevant to, and used in combination with, any other additional layer described herein.

The drug solution layer 430 illustrated in FIG. 4 may be configured to treat painful, swollen, or inflamed injuries, for example an NSAID, such as ibuprofen. The NSAID may be configured for transdermal administration, and may be used in connection with a rate controlling membrane, such as membrane 431, positioned between the drug solution layer 430 and the user's skin. In the illustrated embodiment, rate controlling membranes 431 are shown only on certain of the drug solution layers 430. In some embodiments, there will be no rate controlling membrane 431 associated with the tape, in other embodiments all the drug solution layers 430 may be associated with rate controlling membranes 431, and in still other embodiments certain of the drug solution layers 430 may be associated with rate controlling membranes 431 while others are not. The rate controlling membrane 431 may be configured to control the passage of drug solution through the membrane and onto the skin of the patient. In some embodiments rate controlling membranes 431 with different rate controlling properties may be used simultaneously to allow a higher drug transmission rate on certain drug solution layers 430. Furthermore, the surface area and thickness of the drug solution layer 430 may be configured based on therapy parameters such as desired dose, type of drug, severity and/or location of the injury, and so on. In some embodiments the drug solution layer 430 and/or the rate controlling membrane may be configured to provide treatment for an extended period, for example, up to 24 hours or more. Penetration enhancers, such as, and including chemical penetration enhancement compounds may also be used in connection with the drug solution layer 430 in some embodiments.

Certain chemical modifications or mechanisms can be used to control or influence the delivery of NSAIDs through the skin. For example, different chemical penetration enhancers, sorption promoters or accelerants can be added to the NSAID drug formulation to control the flux of drug through the skin. These enhancers include, for example, the sulphoxides, azones, pyrridones, alchols, alkanols, glycols, durfactants, and terpenes. These compounds and their applications are described further in A. Willians and B. Barry, “Penetration Enhancers,” Advanced Drug Delivery Reviews, vol. 56, pp. 603-618, 2004. Furthermore, in some instances, various concentrations of NSAIDs can be used to control or influence the flux of drug.

It is within the scope of this disclosure to include a rate controlling membrane, such as membrane 431, in connection with drug solution layers in any embodiment disclosed herein. In some embodiments, one base layer may be configured with drug solution layers each of which is associated with a rate controlling membrane, while in other embodiments only certain strips or layers of drug solution may be associated with a rate controlling membrane. Similarly, one base layer may include multiple drug solution layers where different strips of the drug solution are associated with individual membranes which allow diffusion of the drug solution in one strip at a different rate than drug solution in another strip.

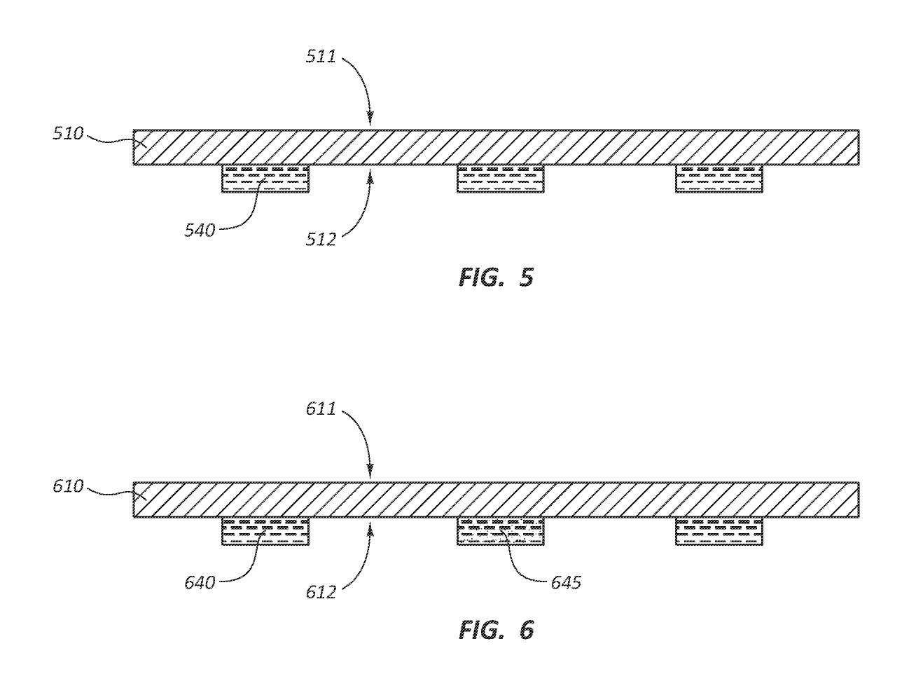

FIG. 5 is a side schematic view of a base layer 510 defining a top surface 511 and a bottom surface 512. In the embodiment of FIG. 5, water channels 540 are shown on the bottom surface 512 of the base layer 510.

In some embodiments, water channels 540, or portions of the bottom surface 512 of the base layer 510 with nothing deposited thereon, may be provided in connection with athletic tape. For example, in an embodiment where portions of the bottom surface 512 have adhesive and/or drug solution layers (not shown), a water channel 540 may be defined as a portion without these additional layers. For convenience in describing the water channels 540, they are illustrated in FIG. 5 in a similar manner to other additional layers in other embodiments. Thus, element 540 of FIG. 5 is intended to schematically illustrate an open portion, not the deposition of a substance on the base layer 510. Water channels provided in connection with other embodiments herein are similarly illustrated.

The water channel 540 may therefore allow the surface of the user's skin to be in direct communication with the bottom surface 512 of the base layer 510. As more fully described in connection with FIGS. 1 and 2, the base layer 510 may be porous in nature; thus the water channel 540 may provide a conduit for water and/or vapor transmission from the surface of the skin across the base layer 510. Water channels 540 may be configured to increase comfort, in particular when a user utilizes the athletic tape for an extended period of time. Thus, in some embodiments, water channels 540 may be provided to minimize or prevent perspiration from being trapped near the skin by the kinesiology tape. In other embodiments, the number or size of the water channels may also, or alternatively, be configured to increase comfort in connection with tape configured for use under water, for example while swimming.

FIG. 6 is a side schematic view of a base layer 610 defining a top surface 611 and a bottom surface 612. In the embodiment of FIG. 6, water channels 640 are provided in connection with water channels that contain a water soluble gel 645. The water soluble gel 645 may be deposited within a portion of the water channels 640. The water soluble gel 645 may be configured to interact with hydrogen bonds within water on the surface of the skin, causing the water to more quickly evaporate off the skin. In some embodiments, gel-containing water channels 640 are interspersed or alternated with open water channels such as 540 of FIG. 5. In some such tapes, every other water channel 640 may contain water soluble gel 645, thus allowing for breathability while also increasing the rate of evaporation.

Further, in some embodiments, the water soluble gel 645 may additionally or alternatively comprise a topical analgesic, such as menthol. In such embodiments, the water soluble gel 645 may increase user comfort due to the interaction of the topical analgesic and the user's skin. Moreover, in some embodiments the water soluble gel 645 may additionally or alternatively comprise aromatic agents such as camphor. In some embodiments camphor may be added to counter the smell of menthol. Additionally, camphor, menthol, or other chemicals may be added to aid in the transdermal delivery of drug solution (430 of FIG. 4) in some embodiments.

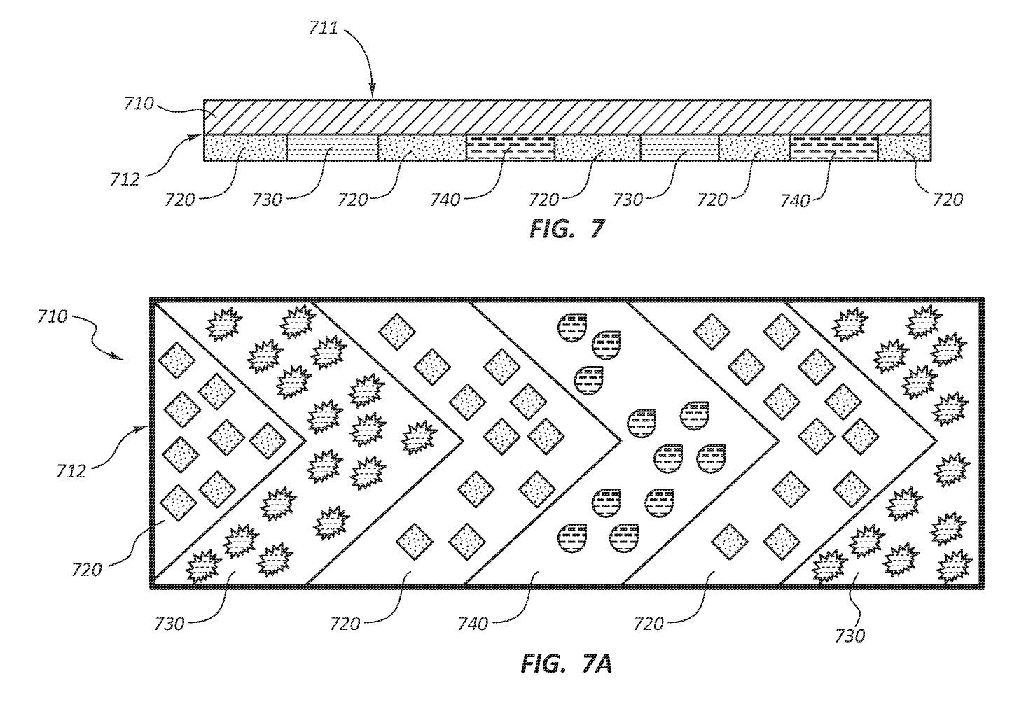

FIG. 7 is a side schematic view of a base layer 710 defining a top surface 711 and a bottom surface 712. In the embodiment of FIG. 7, additional layers are deposited on portions of the bottom surface 712 of the base layer 710 in the following pattern—which may be a repeating pattern and/or used in combination with other patterns—along the length of the base layer 710: an adhesive layer 720, a drug solution layer 730, an adhesive layer 720, and a water channel 740.

FIG. 7A is a bottom schematic view of a portion of the base layer 710 of FIG. 7 showing the relative placement of the additional layers. In the illustrated embodiment, the subsequent layers are arranged in chevron-shaped strips across the width of the base layer 710. In some embodiments, this chevron pattern may be configured to provide moisture control. In the illustrated embodiment, each chevron-shaped strip has about the same width as adjacent strips. In other embodiments, each strip may have a greater or smaller width than any other strip.

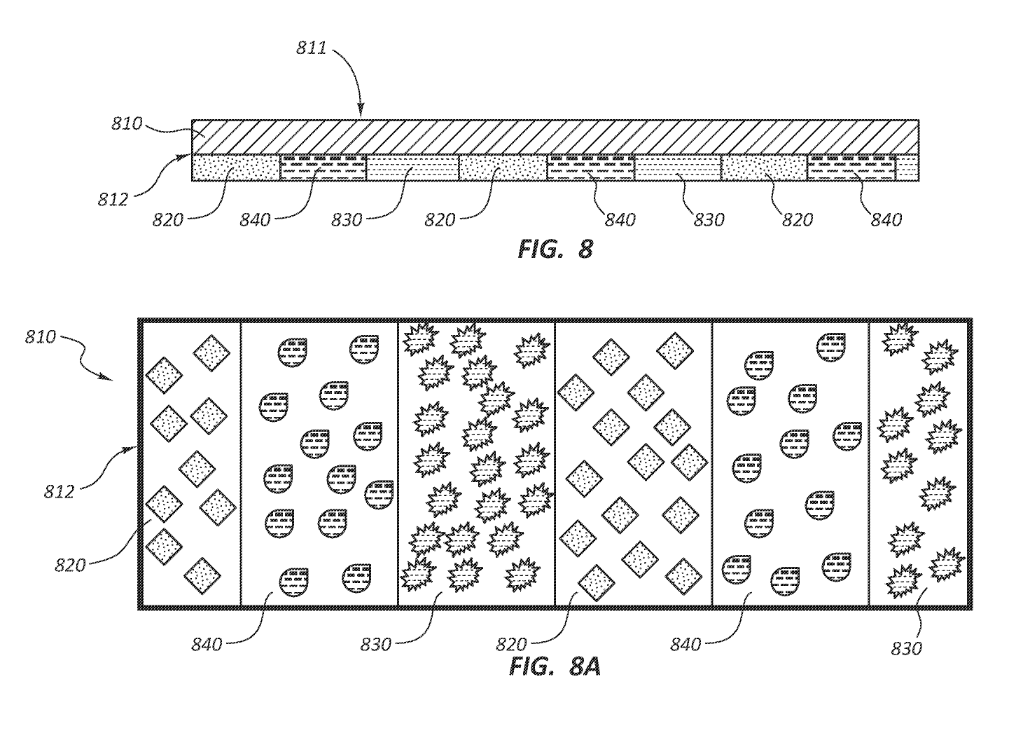

In addition to the chevron pattern described above, in other embodiments, different patterns may be utilized in depositing the additional layers on the base layer 710, including, for example, horizontal lines, vertical lines, circles, polka dots, waves, and so on. In some embodiments the base layers may be arranged in lines or strips which generally run across the width of the base layer, though the strips may or may not be disposed perpendicular to the longitudinal edges of the base layer. Furthermore, in some embodiments each layer may be disposed adjacent at least one other layer on the base layer. FIG. 8 is a side schematic view of a base layer 810 defining a top surface 811 and a bottom surface 812. In the embodiment of FIG. 8, additional layers are deposited on portions of the bottom surface 812 of the base layer 810 in the following pattern—which may be a repeating pattern and/or used in combination with other patterns—along the length of the base layer 810: an adhesive layer 820, a water channel 840, and a drug solution layer 830. The order, relative width, and placement of additional layers may vary depending on factors such as the intended use of the tape, the type of drug utilized in the drug solution layer 830, and so on. Furthermore, some additional layers within the scope of this disclosure may react to other additional layers; in embodiments utilizing such additional layers, the potentially interactive layers may be separated by non-interacting layers.

FIG. 8A is a bottom schematic view of a portion of the base layer 810 of FIG. 8 showing the relative placement of the additional layers. In the illustrated embodiment, the subsequent layers are arranged in parallel strips across the width of the base layer 810. Any of the ordering patterns of additional layers, or the shape or design of those layers, provided in connection with any Figure herein described, is within the scope of the disclosure of any other Figure. Further, any pattern of additional layers disclosed herein may be repeated along the length of a base layer, or may be combined with other patterns to form larger repeating sets of additional layers. Moreover, one base layer may have one or more repeating patterns along a portion of the base layer and other patterns along other portions of the base layer.

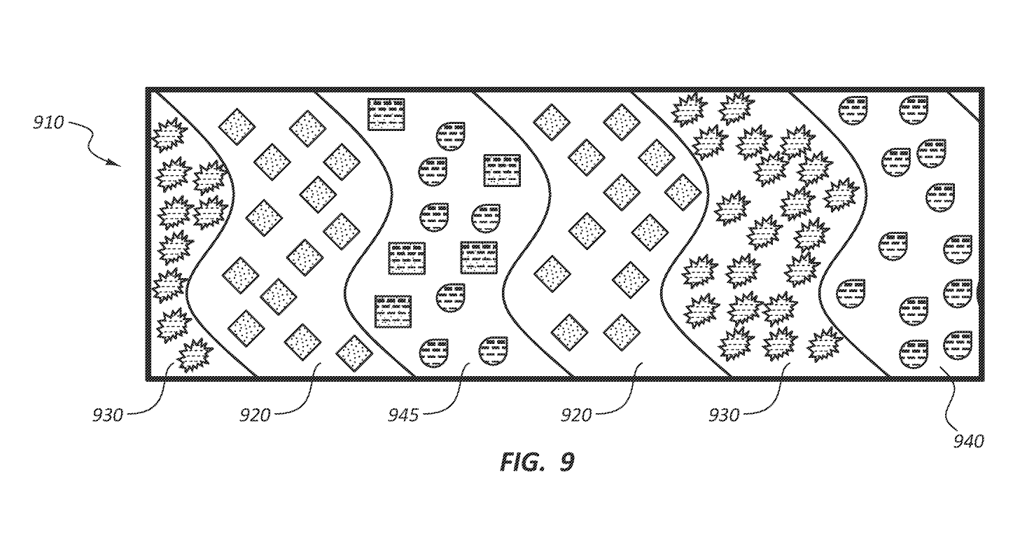

FIG. 9 is a bottom schematic view of a base layer 910, which illustrates another possible arrangement of additional layers on the base layer 910. In the embodiment of FIG. 9, the additional layers, arranged in waves or curved strips across the width of the base layer 910, are deposited in the following order: a drug solution layer 930, an adhesive layer 920, a water channel containing a water soluble gel 945, an adhesive layer 920, a drug solution layer 930, and a water channel 940 (containing no water soluble gel). This pattern may be a repeating pattern and/or used in combination with other pattern.

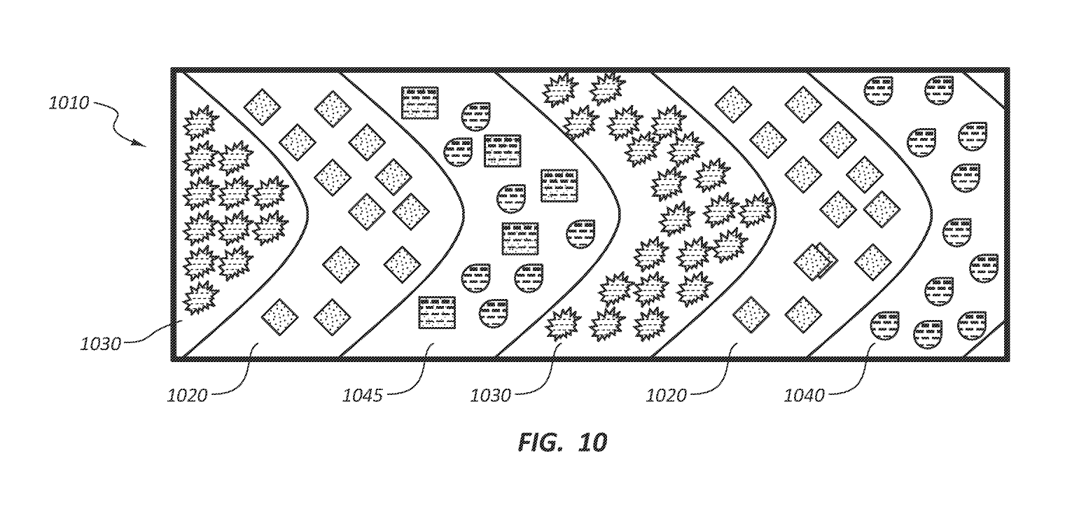

FIG. 10 is a bottom schematic view of another embodiment of a base layer 1010, with additional layers deposited in curved strips in the following pattern: a drug solution layer 1030, an adhesive layer 1020, a water channel containing a water soluble gel 1045, a drug solution layer 1030, an adhesive layer 1020, and a water channel 1040 (containing no water soluble gel). In some embodiments, every other water channel 1040 may contain water soluble gel 1045. In other embodiments other patterns of water soluble gel and water channel layers may be used, such as embodiments where water channels toward the center of the tape contain gel while those near the periphery do not. Any pattern of open water channels in connection with channels containing water soluble gel is within the scope of this disclosure. This pattern may be a repeating pattern and/or used in combination with any other pattern.

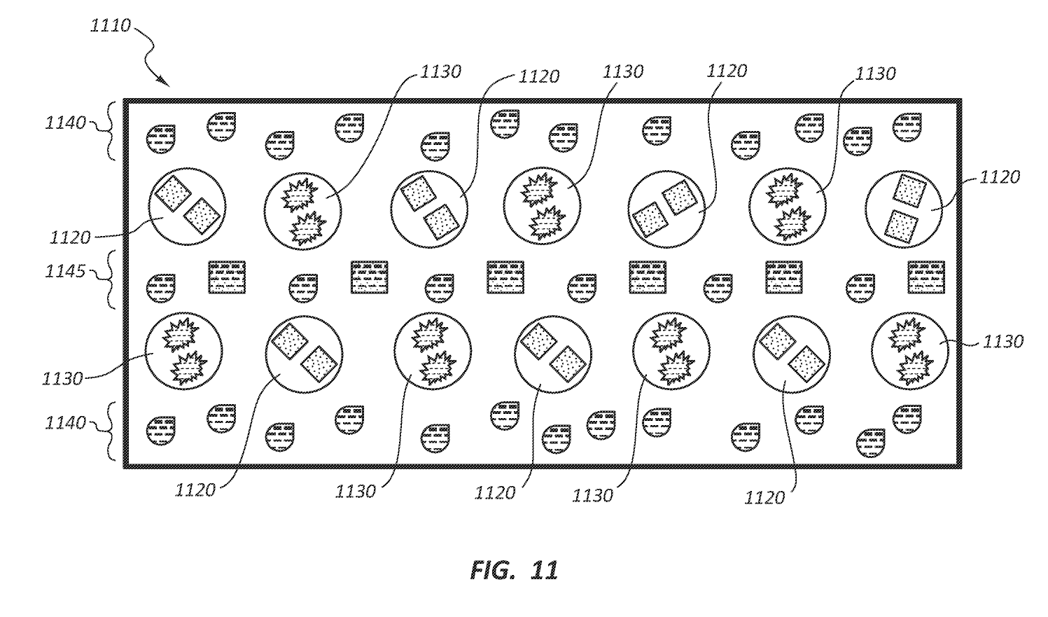

FIG. 11 is a bottom schematic view of a base layer 1110 with additional layers of adhesive 1120 and drug solution 1130 deposited in a dotted pattern on a field of water channels 1140, portions of which contain a water soluble gel 1145. In the illustrated embodiment the adhesive 1120 and drug solution 1130 layers are deposited in an alternating pattern. Other patterns, including random arrangement of the layers, and embodiments wherein the water channels 1140 and/or water channels containing water soluble gel 1145 are also arranged in a dotted-pattern are within the scope of this disclosure.

A variety of sizes of athletic or other treatment tape are within the scope of this disclosure. Design parameters such as length and width may depend, for example, on the intended use of the tape. Tape may be provided as a roll from which strips may be cut or torn to a desired size and subsequently applied in a desired pattern, or alternately, provided in pre-sized, pre-shaped and/or pre-configured shapes or sizes for use in specified applications. Tape within the scope of this disclosure may be configured for use as athletic tape, tape to treat soft tissue injuries, tape to treat sports injuries, tape to treat in post surgery recovery, and so on. In some embodiments a backing material may be provided in connection with the tape to cover and protect the adhesive layers and other additional layers during manufacturing, shipping, storage, etc.

The examples and embodiments disclosed herein are to be construed as merely illustrative and exemplary, and not as a limitation of the scope of the present disclosure in any way. It will be apparent to those having skill in the art with the aid of the present disclosure that changes may be made to the details of the above-described embodiments without departing from the underlying principles of the disclosure herein.

1. Athletic tape configured for application to the human skin, comprising:
a base layer, defining a top surface and a bottom surface, the base layer configured to provide sufficient support and strength to decrease pressure on muscles by lifting the skin; and
a layer of drug solution deposited on a portion of the bottom surface of the base layer, the drug configured to be transdermally administered.

2. The athletic tape of claim 1, wherein the drug solution comprises an NSAID.

3. The athletic tape of claim 1, further comprising a rate controlling membrane configured to be disposed between the drug solution and the skin of a user.

4. The athletic tape of claim 1, wherein the base layer is comprised of woven cotton fibers.

5. The athletic tape of claim 1, further comprising one or more water channels, comprising portions wherein the base layer is configured to be in direct communication with the skin of a user.

6. The athletic tape of claim 5, wherein the water channels are configured to promote vapor exchange across the base layer.

7. The adhesive tape of claim 5, wherein one or more water channels comprise layers of water soluble gel deposited on a portion of the bottom surface.

8. The adhesive tape of claim 1, further comprising an adhesive layer deposited on a portion of the bottom surface.

9. The adhesive tape of claim 8, wherein drug solution layers and adhesive layers are arranged in chevron-shaped strips running across the width of the base layer.

10. The adhesive tape of claim 9, further comprising water channels arranged in chevron-shaped strips running across the width of the base layer.

11. The adhesive tape of claim 10, wherein the chevron-shaped strips are arranged in a repeating pattern of: an adhesive layer, a drug solution layer, an adhesive layer, and a water channel.

12. The adhesive tape of claim 10, wherein the chevron-shaped strips are arranged in a repeating pattern of: an adhesive layer, a water channel, and a drug solution layer.

13. The adhesive tape of claim 10, further comprising water soluble gel layers arranged in chevron-shaped strips running across the width of the base layer.

14. The adhesive tape of claim 13, wherein the chevron-shaped strips are arranged in a repeating pattern of: a drug solution layer, an adhesive layer, a water soluble gel layer, an adhesive layer, a drug solution layer, and a water channel.

15. The adhesive tape of claim 13, wherein the chevron-shaped strips are arranged in a repeating pattern of: a drug solution layer, an adhesive layer, a water soluble gel layer, a drug solution layer, an adhesive layer, and a water channel.

16. The adhesive tape of claim 8, wherein drug solution layers and adhesive layers are arranged in wave-like strips running across the width of the base layer.

17. The adhesive tape of claim 8, wherein drug solution layers and adhesive layers are arranged in parallel strips running across the width of the base layer.

18. Athletic tape configured for application to the human skin, comprising:
a base layer, defining a top surface and a bottom surface, the base layer configured to provide sufficient support and strength to decrease pressure on muscles by lifting the skin; and
a plurality of additional layers disposed on the bottom surface of the base layer, each additional layer of the plurality of additional layers disposed adjacent at least one other additional layer on the bottom surface of the base layer,
wherein the additional layers are disposed such that no additional layer is disposed directly between another additional layer and the base layer.

19. The athletic tape of claim 18, wherein the additional layers are selected from the group consisting of: layers of adhesive, layers of drug solution, open layers configured to promote vapor transfer across the base layer, and layers of water soluble gel.

20. The athletic tape of claim 18, wherein the additional layers are disposed in parallel strips running across the width of the base layer.
